SEQUENCE LISTING (15 sequences)

SEQ ID NO: 1 - protein, 12 aa, Artificial Sequence
  bovine lactoferrin peptide from 418 aa to 429 aa
SKHSSLDCVLRP

SEQ ID NO: 2 - protein, 17 aa, Artificial Sequence
  bovine lactoferrin peptide from 506 aa to 522 aa
AGDDQGLDKCVPNSKEK

SEQ ID NO: 3 - protein, 11 aa, Artificial Sequence
  bovine lactoferrin peptide from 553 aa to 563 aa
NGESTADWAKN

SEQ ID NO: 4 - protein, 11 aa, Artificial Sequence
  modified SEQ ID NO3 wherein the threonine residue is substituted by a homologue serine
NGESSADWAKN

SEQ ID NO: 5 - protein, 348 aa, Artificial Sequence
  bovine lactoferrin C-lobe peptide
YTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 6 - protein, 18 aa, Artificial Sequence
  first 18 aminoacids of N-terminus sequence corresponding to the highly conserved fusion peptide of viral hemagglutinin (HA) HA2 subunit of H1N1 Solomon
GLFGAIAGFIEGGWTGMV

SEQ ID NO: 7 - protein, 341 aa, Artificial Sequence
  Bovine lactoferrin N-lobe peptide
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKAR

SEQ ID NO: 8 - protein, 14 aa, Artificial Sequence
  bovine lactoferrin peptide from 441 aa to 454 aa
KANEGLTWNSLKDK

SEQ ID NO: 9 - protein, 23 aa, Artificial Sequence
  peptide of bovine lactoferrin C-lobe from aa 478 to aa 501
TGSCAFDEFFSQSCAPGADPKSR

SEQ ID NO: 10 - protein, 12 aa, Artificial Sequence
  bovine lactoferrin C-lobe peptide from aa 619 to aa 630
GKNGKNCPDKFC

SEQ ID NO: 11 - protein, 6 aa, Artificial Sequence
  bovine lactoferrin C-lobe peptide from aa 633 to aa 637
KSETKN

SEQ ID NO: 12 - protein, 18 aa, Artificial Sequence
  bovine lactoferrin C-lobe peptide from aa 642 to aa 659
NDNTECLAKLGGRPTYEE

SEQ ID NO: 13 - protein, 5 aa, Artificial Sequence
  bovine lactoferrin C-lobe peptide from aa 425 to aa 429
CVLRP

SEQ ID NO: 14 - protein, 4 aa, Artificial Sequence
  bovine lactoferrin C-lobe peptide from aa 426 to aa 429
VLRP

SEQ ID NO: 15 - protein, 11 aa, Artificial Sequence
  bovine lactoferrin C-lobe peptide from aa 649 to aa 659
AKLGGRPTYEE

(19) United States
(12) Patent Application Publication
(10) Pub. No.: US 2014/0322196 A1
(43) Pub. Date: Oct. 30, 2014

(54) LACTOFERRIN DERIVED PEPTIDES FOR USE AS BROAD-SPECTRUM INHIBITORS OF INFLUENZA VIRUS INFECTION

(71) Applicants: ISTITUTO SUPERIORE DI SANITA', ROMA, RM (IT); UNIVERSITA' DEGLI STUDI "G. D'ANNUNZIO", CHIETI (IT)

(72) Inventors: Fabiana E.D. Superti, Roma (IT); Mariangela Agamennone, Chieti (IT); Maria Grazia Ammendolia, ROMA (IT); Agostina Pietrantoni, Roma (IT); Fabio Lannutti, Chieti (IT)

(21) Appl. No.: 14/358,713

(22) PCT Filed: Nov. 16, 2012
(86) PCT No.: PCT/IT2012/000349

§ 371 Date: May 15, 2014

(30) Foreign Application Priority Data
Nov. 16, 2011 (IT) RM2011A000606

(51) Int. Cl. C12N 9/64; G01N 33/50; A61K 38/48

(52) U.S. Cl. CPC C12N 9/6424; A61K 38/482; G01N 33/502
USPC 424/94.64; 435/226; 435/5

(57) ABSTRACT

The present disclosure describes lactoferrin C-lobe and peptides derived thereof used as broad spectrum inhibitors of Influenza virus group 1 and 2 hemagglutination and infection.

The present invention concerns lactoferrin derived peptides for use as broad-spectrum inhibitors of Influenza virus infection. Particularly, the present invention concerns lactoferrin C-lobe derived peptides, preferably bovine lactoferrin C-lobe derived peptides, for use as, broad-spectrum inhibitors of Influenza virus infection able to inhibit hemagglutination and infection of cells by Influenza virus belonging to all major subtypes including H1N1 and H3N2.

Influenza virus infections are a major risk to human safety and animal health worldwide. Viral Influenza disease represents a serious source of morbidity and mortality worldwide and a considerable cause of illness and death among people with immunodeficiency associated with aging or different clinical conditions. Control of Influenza is essentially based on vaccines and few antiviral drugs. While vaccines are the core measure for infection control, the immunization programs are not fully effective because of rapid virus antigenic drift, so that vaccine antigen composition needs to be updated annually based on global influenza surveillance. Efforts to Influenza prevention by vaccination are made difficult by the virus ability to rapidly mutate and recombine into antigenically new viral particles, sometimes leading to the emergence of a totally new virus.

Antiviral chemotherapy is based on two classes of drugs: amantadine and its derivative rimantadine (Dolin et al., 1982) and neuraminidase inhibitors (oseltamivir and zanamivir) (Smith et al., 2011). Amantadine and derivatives prevent viral uncoating by blocking the ion channel function of the M2 protein of the Influenza virus. These drugs reduce the duration of symptoms of clinical influenza, but major side effects and the emergence of drug-resistant variants have been described (Hayden 2006; Fiore et al., 2008). Consequently, amantadine and derivatives are no longer the first choice for Influenza therapy. Neuraminidase inhibitors prevent viral budding from infected cells by inhibiting the cleavage of host sialic acid residues and remain, at present, the primary treatment against Influenza. However, they have limited efficacy if administered late in infection and widespread use is likely to result in the emergence of resistant viral strains (Kiso et al., 2004; Dharan et al., 2009; Cheng et al., 2010).

The availability of broad-spectrum antiviral drugs is an important asset in the fight against influenza. Particularly, a combination of different anti-Influenza drugs, each directed against a different viral target or different mode of action, would be expected to be more active in Influenza treatment and also in minimizing drug resistance. In fact, combination of amantadine and oseltamivir has been shown to decrease the emergence of drug-resistant Influenza A viruses (Ilyushina et al., 2006). Although this has not been observed in all cases.

On the basis of the above it is therefore apparent the need to provide for new compounds against Influenza virus able to overcome the disadvantages of the known therapies.

New therapeutic strategies are therefore a universal public health priority. An attractive antiviral strategy is the blocking of Influenza virus entry into the host cell. This process is mediated by the viral hemagglutinin (HA), which is responsible for the binding of the virus to the target cell and, after virus uptake into endosomes, fusion of the virus with the cell membranes (Skehel and Wiley, 2000). The HA of Influenza is the major glycoprotein component of the viral envelope. HA is homotrimeric and is composed of two polypeptide segments, designated HA₁ and HA₂, attached to each other via a disulfide linkage (Wilson et al., 1981). The HA₁ segments mediate HA attachment to the host cell surface by binding to sialic acid-containing cell surface glycans. After attachment, virions internalised by endocytoses undergoes an irreversible acid-induced structural rearrangement in which the highly hydrophobic amino terminus of HA₂ is exposed. This hydrophobic fusion peptide is then translocated toward the endosomal membrane mediating fusion of the viral envelope with the cell membranes and formation of a fusion pore (Wiley and Skehel, 1987). This conformational change is crucial for the fusogenic activity of HA and for viral entry. Coupled with the fact that the hydrophobic fusion peptide is the only universally conserved epitope in all Influenza viruses, it represents a very attractive target for novel anti-Influenza drugs.

Breast-feeding has been recognised to protect against respiratory and gastrointestinal infections in infants (May, 1988). Milk, besides secretory IgA and IgM, also contains a number of various non-antibody components with known antimicrobial activity, including lactoferrin (May, 1988; Levay and Viljoen, 1995). Lactoferrin (Lf) is an 80-kDa multifunctional cationic glycoprotein belonging to the transferrin family (Levay and Viljoen, 1995). It is present in mucosal secretions, such as tears, saliva, nasal exudate, gastrointestinal fluids, seminal and vaginal fluids, and in granules of polymorphonuclear leukocytes (Levay and Viljoen, 1995). Lf possesses a variety of biological functions such as: influence on iron homeostasis, immunomodulation, and inhibitory activity towards different pathogens (Levay and Viljoen, 1995; Valenti and Antonini, 2005). Bovine lactoferrin (bLf) has been recognized as potent inhibitor of different enveloped viruses such as human cytomegalovirus (Harmsen et al., 1995), Herpes Simplex Viruses types 1 and 2 (Marchetti at al., 1996, 2004, 2009; Ammendolia et al., 2007a), human immunodeficiency virus (HIV) (Harmsen et al., 1995; Swart et al., 1996; Berkhout et al., 2002, 2004), human hepatitis C virus (Ikeda et al., 2000), hantavirus (Murphy at al., 2000), hepatitis B virus (Hara et al., 2002), respiratory syncytial virus (Sano et al., 2003), Sindbis and Semliki Forest viruses (Waarts et al., 2005). BLf, similarly to lactoferrin of other mammalian species, like human lactoferrin, is a glycoprotein folded in two symmetrical lobes (N- and C-lobes), with high sequence homology, possibly resulting from an ancestral gene duplication (Norris et al., 1986; Anderson et al., 1987). Each lobe further consists of two sub-lobes or domains called N1, N2, C1 and C2, respectively. In bovine lactoferrin, the N1 stands for the sequences 1-90 and 251-333, N2 for 91-250, C1 for 345-431 and 593-676, and C2 for 432-592 (Moore et al., 1997; Steijns and van Hooijdonk, 2000). The above domains delimit a Fe³⁺ binding site (Norris et al., 1986; Moore et al., 1997). Although it is generally accepted that, with few exceptions, the inhibiting activity of lactoferrin takes place in the early phases of viral infection, its antiviral effect seems to be exercised in different ways among different viral species. The principal suggested mechanisms for the antiviral activity are a direct binding of lactoferrin to viral particles (Swart et al., 1996; Superti et al., 1997; Marchetti et al., 1999; Pietrantoni et al., 2003; Ammendolia et al., 2007b) or to host cell molecules that the virus uses as a receptor or a co-receptor (Marchetti et al., 1996, 2004; Andersen et al., 2001; Di Biase et al., 2003). An additional effect of bLf on a later intracellular step of virus infection has been described (Superti et al., 1997; Tinari et al., 2005) and, more recently, the inventors of the present invention demonstrated that bLf treatment is able to prevent Influenza virus-induced programmed cell death by interfering with function of caspase 3 (Pietrantoni et al., 2010). This caused the block of nuclear export of viral ribonucleoproteins with consequent viral assembly prevention.

In an effort to identify new antiviral therapies effective against Influenza virus, according to the present invention, the inventors have attempted to deeper investigate the mechanism of the anti-Influenza virus effect of bLf and the role of its tryptic fragments (the N- and C-lobes) in the antiviral activity. In particular, they have evaluated the influence of bLf on hemagglutinin-mediated functions. Hemagglutinin has been chosen since it is the major surface protein of the Influenza A virus and is essential to the entry process so representing an attractive target for antiviral therapy. An initial attachment of HA to specific receptors on the host cell surface and a membrane fusion of HA matured by protease digestion are required for virus infection. As a matter of fact, neutralizing compounds targeting HA represent a useful tool in neutralizing viral infection, clearing virus, and suppressing viral spread. Results obtained indicated that lactoferrin is able to bind the HA₂ fragment of all Influenza A viruses tested. In particular, the activity of the C-lobe was comparable to that of the entire protein, in the ability to prevent both viral hemagglutination and infection. Importantly, some peptides derived from C-lobe were able to prevent both viral hemagglutination and infection to a much higher potency as compared to the entire protein.

Interestingly, lactoferrin was shown to bind to the HA₂ subunit of viral HA, particularly to the fusion peptide, the only universally conserved epitope in all Influenza virus hemagglutinin. This has been demonstrated by N-terminus protein sequencing of subunit samples and by ELISA tests in which the fusion peptide was sufficiently exposed to allow access to bLf. To our knowledge, this is the first demonstration that viral hemagglutination can be inhibited by a specific interaction with the HA₂ subunit. As a matter of fact, neutralizing antibodies against Influenza virus have been found to act by two different mechanisms, mirroring the dual functions of hemagglutinin: (i) prevention of attachment to target cells, (ii) inhibition of entry (membrane fusion). The mechanism of neutralization depends on the HA site recognized by the HA-specific molecule. Usually, antibodies against HA act by inhibiting attachment. This occurs as they bind HA₁ and physically hinder the interaction with sialic acid receptors on target cells (Edwards and Dimmock, 2001). Some antibodies have been found to prevent membrane fusion; most of them recognize sites in the HA₂ region, far away from the receptor-binding site (Ekiert et al., 2009; Sui et al., 2009).

Type A influenza viruses are serologically divided into 16 HA subtypes which are further divided into two major phylogenetic groups: group 1 (subtypes H1, H2, H5, H6, H8, H9, H11, H12, H13, and H16) and group 2 (subtypes H3, H4, H7, H10, H14, and H15). This separation of the 16 subtypes correlates with two distinct basic structures taken by the stalk of the HA (Ekiert et al., 2009). BLf shows activity against viruses expressing hemagglutinin proteins from both group 1 and group 2 as it is able to neutralize isolates from 4 of the 16 influenza A subtypes: H1, H3, H5, and H7. Inventors' working hypothesis is that the interaction of bLf with the highly conserved region of HA (fusion peptide) could allow to a “conformational change” of hemagglutinin so preventing both hemagglutination and infection.

Protein-protein docking calculations, performed to find a putative binding mode of bLf C-lobe to HA, demonstrated the possibility of the bLf targeting a conserved region of HA, close to the fusion peptide. This HA region shares common feature with that of other viral strains, suggesting the possibility to target this part of the hemagglutinin stem for broad spectrum anti-influenza drug design. These calculations provided strong indication about the role of three loops of bLf C-lobe in the interaction with HA, corresponding to the sequences 506-522, 418-429 and 553-563. The two latter sequences are not present in the N-lobe of bLf, which in fact resulted inactive, and the first one presents the 29% of identity with respect to the corresponding portion of the N-lobe. Therefore, three main peptides were identified: SKHSSLDCVLRP (418-429) (SEQ ID NO: 1), AGDDQGLDKCVPNSKEK (506-522) (SEQ ID NO: 2) and NGESTADWAKN (553-563) (SEQ ID No: 3). Because of poor stability of SEQ ID NO:3 peptide, a modified sequence has been designed where the threonine residue is substituted by a homologue serine obtaining a peptide of sequence NGESSADWAKN (SEQ ID NO: 4).

Protein-protein docking calculations suggested the possible role of other loops of bLf C-lobe that can contribute to the binding to HA, corresponding to the sequences 441-454, 478-501, 619-630, 633-637 and 642-659. These loops, like the above mentioned peptides, are bLf solvent exposed regions, located on the C-lobe surface properly to interact with the HA stem region, with a sequence similarity lower than 33% with respect to N-lobe. These peptide are: KANEGLTWNSLKDK (441-454) (SEQ ID NO: 8), TGSCAFDEFFSQSCAPGADPKSR (478-501) (SEQ ID NO: 9), GKNGKNCPDKFC (619-630) (SEQ ID NO: 10), KSETKN (633-637) (SEQ ID NO: 11), NDNTECLAKLGGRPTYEE (642-659) (SEQ ID NO: 12).

The above peptides with SEQ ID NO: 1, 2 and 4 were synthesized and tested for their ability to inhibit hemagglutination and infection.

The results showed that lactoferrin-derived peptides bind to HA and neutralize hemagglutination and in vitro infection of different strains of Influenza virus with a very high potency, suggesting that these bLf regions were indeed involved in the bLf inhibition of Influenza virus. Importantly, the peptides are active on viral subtypes belonging to the two major phylogenetic groups of Influenza virus. These peptides may represent a valuable tool for the study of influenza viruses and their receptor-binding interactions, as well as for therapeutic possibilities, that will be examined in in vivo tests. The short sequence is an added advantage for both metabolic stability and commercialisation purpose as it can greatly reduce the cost of production.

Therefore, it is a specific object of the present invention a peptide of lactoferrin C-lobe (i.e. a fragment of lactoferrin C-lobe), homolog peptide thereof wherein at least one amino acid is substituted with a corresponding homolog amino acid, wherein homolog amino acid is defined as one with functionally equivalent physicochemical properties, both natural and non-natural, fragment of said peptide or said homolog peptide, or mixture of at least one of said peptide and/or said homolog peptide and/or said fragment, said peptide being a solvent exposed loop region of lactoferrin C-lobe and having percentage of identity lower than 33% with respect to a corresponding peptide of lactoferrin N-lobe after optimal alignment; wherein said fragment of said peptide or homolog peptide is different from CVLRP (SEQ ID NO: 13), VLRP (SEQ ID NO: 14), CVL, RP, VL, AKLGGRPTYEE (SEQ ID NO: 15). The lactoferrin C-lobe can be a bovine, zebu, wild yak, cow buffalo, goat or sheep lactoferrin C-lobe.

Particularly, the present invention concerns a peptide of lactoferrin C-lobe, homolog peptide thereof, fragment of said peptide or homolog peptide or mixture of at least one of said peptide and/or said homolog peptide and/or said fragment according to the above, wherein said peptide is chosen from the group consisting of SKHSSLDCVLRP (418-429) (SEQ ID NO: 1), AGDDQGLDKCVPNSKEK (506-522) (SEQ ID NO: 2), NGESSADWAKN (SEQ ID NO: 4), NGESTADWAKN (553-563) (SEQ ID No: 3), KANEGLTWNSLKDK (441-454) (SEQ ID NO: 8), TGSCAFDEFFSQSCAPGADPKSR (478-501) (SEQ ID NO: 9), GKNGKNCPDKFC (619-630) (SEQ ID NO: 10), KSETKN (633-637) (SEQ ID NO: 11), NDNTECLAKLGGRPTYEE (642-659) (SEQ ID NO: 12).

The peptide having sequence SEQ ID NO:1 is present in bovine, wild yak, zebu or cow buffalo lactoferrin C-lobe, SEQ ID NO:2 is a peptide of bovine, zebu, cow buffalo or goat lactoferrin C-lobe, SEQ ID NO: 4 is a peptide of goat or sheep lactoferrin C-lobe or a modified sequence of bovine lactoferrin C-lobe peptide NGESTADWAKN (SEQ ID No: 3) wherein the threonine residue is substituted by a homologue serine. The peptide having sequence SEQ ID NO:3 is present in bovine, wild yak, zebu or cow buffalo lactoferrin C-lobe, SEQ ID NO:8 is a peptide of bovine, wild yak or zebu lactoferrin C-lobe, SEQ ID NO:9 is a peptide of bovine, wild yak, zebu, cow buffalo, goat or sheep lactoferrin C-lobe, SEQ ID NO:10 is a peptide of bovine, wild yak or zebu lactoferrin C-lobe, SEQ ID NO:11 is a peptide of bovine, wild yak, zebu, cow buffalo, goat and sheep lactoferrin C-lobe, SEQ ID NO:12 is a peptide of bovine, wild yak, zebu, cow buffalo, goat or sheep lactoferrin C-lobe.

Wild yak, zebu, cow buffalo, goat and sheep lactoferrin C-lobes have identity percentage in comparison to bovine lactoferrin C-lobe of 99.1%, 98.6%, 97.4%, 95.7% and, 94.5%, respectively. SEQ ID NO: 1, 2, 3, 4, 8, 9, 10, 11 and 12 have been found in the above specified lactoferrins of previously specified species with 100% identity meanwhile they have not been found in other species.

However, the present invention refers to lactoferrin C-lobe sequence or fragments thereof both prepared by synthesis and extracted by natural source.

The present invention concerns also a peptide of lactoferrin C-lobe, homolog peptide thereof, fragment of said peptide or homolog peptide or mixture of at least one of said peptides and/or said homolog peptide and/or said fragment as defined above, for use as a medicament.

Therefore, the present invention concerns a pharmaceutical composition comprising or consisting of the peptide of lactoferrin C-lobe, homolog peptide thereof, fragment of said peptide or homolog peptide or mixture of at least one of said peptide and/or said homolog peptide and/or said fragment, as defined above, as active principle, together with one or more excipients and/or adjuvant pharmaceutically acceptable.

It is further object of the present invention a peptide of lactoferrin C-lobe (i.e. a fragment of lactoferrin C-lobe), homolog peptide thereof wherein at least one amino acid is substituted with a corresponding homolog amino acid, wherein homolog amino acid is defined as one with functionally equivalent physicochemical properties, both natural and non-natural, fragment of said peptide or homolog peptide, or mixture of at least one of said peptide and/or said homolog peptide and/or said fragment, or lactoferrin C-lobe (i.e. the entire lactoferrin C-lobe) or mixtures of said lactoferrin C-lobe with at least one of said peptide and/or said homolog peptide and/or said fragment, or pharmaceutical composition as defined above, for use in the treatment of Influenza virus infection, for instance a type A Influenza virus infection, wherein said peptide is a solvent exposed loop region of lactoferrin C-lobe and has percentage of identity lower than 33% with respect to a corresponding peptide of lactoferrin N-lobe after optimal alignment.

The lactoferrin C-lobe can be a bovine, wild yak, zebu cow buffalo, goat or sheep lactoferrin C-lobe.

Type A Influenza virus infection comprises H1, H2, H5, H6, H8, H9, H11, H12, H13, H16, H3, H4, H7, H10, H14, and H15 subtypes.

Particularly, the present invention concerns a peptide of lactoferrin C-lobe, homolog peptide thereof wherein at least one amino acid is substituted with a correspondent homolog amino acid (as defined above), fragment of said peptide or homolog peptide, or mixture of at least one of said peptide and/or said homolog peptide and/or said fragment, or lactoferrin C-lobe or mixtures of said lactoferrin C-lobe with at least one of said peptide and/or said homolog peptide and/or said fragment, or pharmaceutical composition as defined above, for use in the treatment of Influenza virus infection, wherein said peptide comprises or consists of a peptide of lactoferrin C-lobe chosen from the group consisting of SKHSSLDCVLRP (SEQ ID NO: 1), AGDDQGLDKCVPNSKEK (SEQ ID NO: 2), NGESSADWAKN (SEQ ID NO: 4), NGESTADWAKN (SEQ ID No: 3), KANEGLTWNSLKDK (441-454) (SEQ ID NO: 8), TGSCAFDEFFSQSCAPGADPKSR (478-501) (SEQ ID NO: 9), GKNGKNCPDKFC (619-630) (SEQ ID NO: 10), KSETKN (633-637) (SEQ ID NO: 11), NDNTECLAKLGGRPTYEE (642-659) (SEQ ID NO: 12). Preferably, the peptide is chosen among SKHSSLDCVLRP (SEQ ID NO: 1), AGDDQGLDKCVPNSKEK (SEQ ID NO: 2), NGESSADWAKN (SEQ ID NO: 4).

As mentioned above, the peptide having sequence SEQ ID NO:1 is present in bovine, wild yak, zebu or cow buffalo lactoferrin C-lobe, SEQ ID NO:2 is a peptide of bovine, zebu, cow buffalo or goat lactoferrin C-lobe, SEQ ID NO: 4 is a peptide of goat or sheep lactoferrin C-lobe or a modified sequence of bovine lactoferrin C-lobe peptide NGESTADWAKN (SEQ ID No: 3) wherein the threonine residue is substituted by a homologue serine. The peptide having sequence SEQ ID NO:3 is present in bovine, wild yak, zebu or cow buffalo lactoferrin C-lobe, SEQ ID NO:8 is a peptide of bovine, wild yak or zebu lactoferrin C-lobe, SEQ ID NO:9 is a peptide of bovine, wild yak, zebu, cow buffalo, goat or sheep lactoferrin C-lobe, SEQ ID NO:10 is a peptide of bovine, wild yak or zebu lactoferrin C-lobe, SEQ ID NO:11 is a peptide of bovine, wild yak, zebu, cow buffalo, goat and sheep lactoferrin C-lobe, SEQ ID NO:12 is a peptide of bovine, wild yak, zebu, cow buffalo, goat or sheep lactoferrin C-lobe.

According to the present invention, the lactoferrin C-lobe can be a bovine lactoferrin C-lobe consisting of the following sequence SEQ ID No:5:

YTRVVWCAVGPEEQKKCQQWSQQSGQNVTCATASTTDDCIVLVLKGEADA
LNLDGGYIYTAGKCGLVPVLAENRKSSKHSSLDCVLRPTEGYLAVAVVKK
ANEGLTWNSLKDKKSCHTAVDRTAGWNIPMGLIVNQTGSCAFDEFFSQSC
APGADPKSRLCALCAGDDQGLDKCVPNSKEKYYGYTGAFRCLAEDVGDVA
FVKNDTVWENTNGESTADWAKNLNREDFRLLCLDGTRKPVTEAQSCHLAV
APNHAVVSRSDRAAHVKQVLLHQQALFGKNGKNCPDKFCLFKSETKNLLF
NDNTECLAKLGGRPTYEEYLGTEYVTAIANLKKCSTSPLLEACAFLTR.

In addition the present invention concerns the use of a peptide of lactoferrin C-lobe, homolog peptide thereof wherein at least one amino acid is substituted with a correspondent homolog amino acid, wherein homolog amino acid is defined as one with functionally equivalent physicochemical properties, both natural and non-natural, fragment of said peptide or homolog peptide, according to the above, as a tool for study evolution of influenza virus and receptor-binding interaction. Particularly, said peptide comprises or consists of a peptide (of lactoferrin C-lobe) chosen from the group consisting of SKHSSLDCVLRP (SEQ ID NO: 1), AGDDQGLDKCVPNSKEK (SEQ ID NO: 2), NGESSADWAKN (SEQ ID NO: 4), NGESTADWAKN (SEQ ID No: 3), KANEGLTWNSLKDK (441-454) (SEQ ID NO: 8), TGSCAFDEFFSQSCAPGADPKSR (478-501) (SEQ ID NO: 9), GKNGKNCPDKFC (619-630) (SEQ ID NO: 10), KSETKN (633-637) (SEQ ID NO: 11), NDNTECLAKLGGRPTYEE (642-659) (SEQ ID NO: 12).

The present invention now will be described, by illustrative and not limitative way, according to preferred embodiments thereof with particular reference to enclosed drawings wherein:

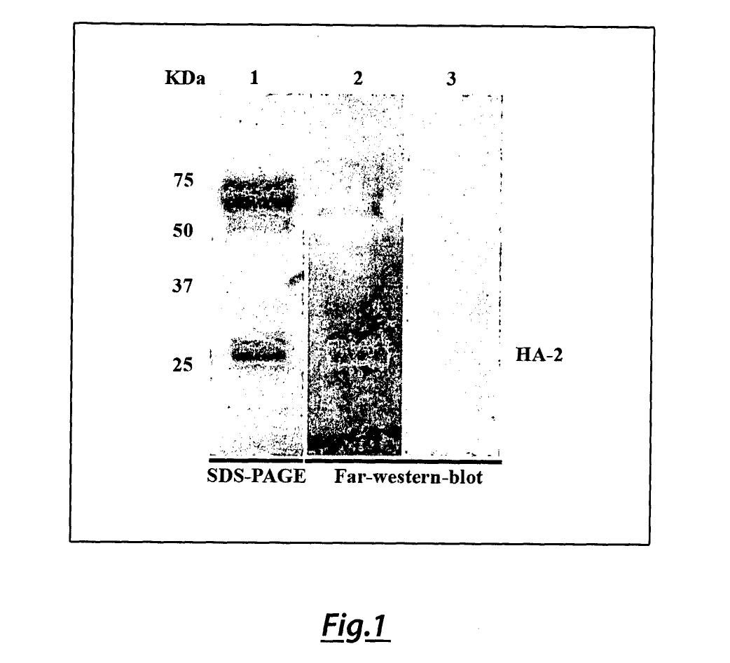

FIG. 1 shows ligand-blot assay of bLf binding to Influenza A Virus H1N1 subunits. Lane 1: SDS-PAGE (Sodium Dodecyl Sulphate-PolyAcrylamide Gel Electrophoresis) of reduced A/Solomon 3/2006 H1N1 subunits. Lane 2: lactoferrin blot overlay. Lane 3: specificity control (rabbit anti-Lf antibodies+horseradish peroxidase (HRP)-conjugated anti-rabbit antibodies).

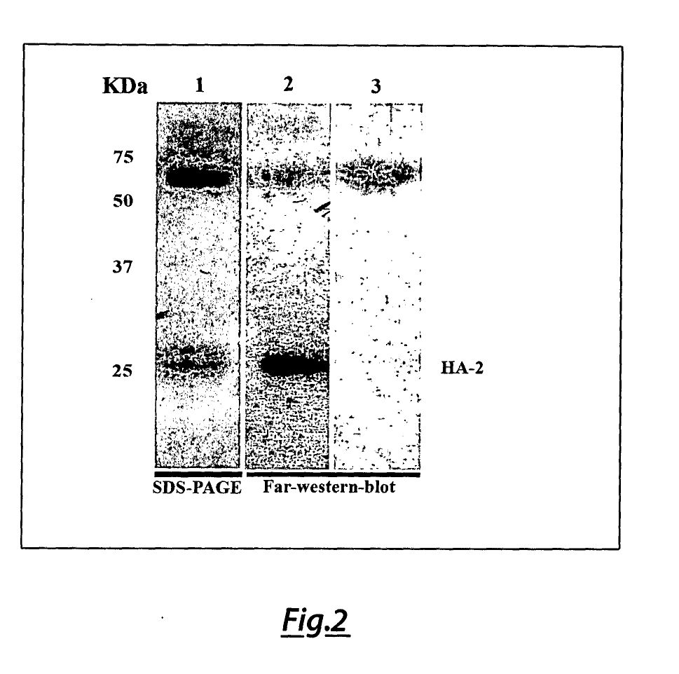

FIG. 2 shows ligand-blot assay of bLf binding to Influenza A Virus H3N2 subunits. Lane 1: SDS-PAGE of reduced A/Wisconsin (67/05) H3N2 subunits. Lane 2: lactoferrin blot overlay. Lane 3: specificity control (rabbit anti-Lf antibodies+HRP-conjugated anti-rabbit antibodies).

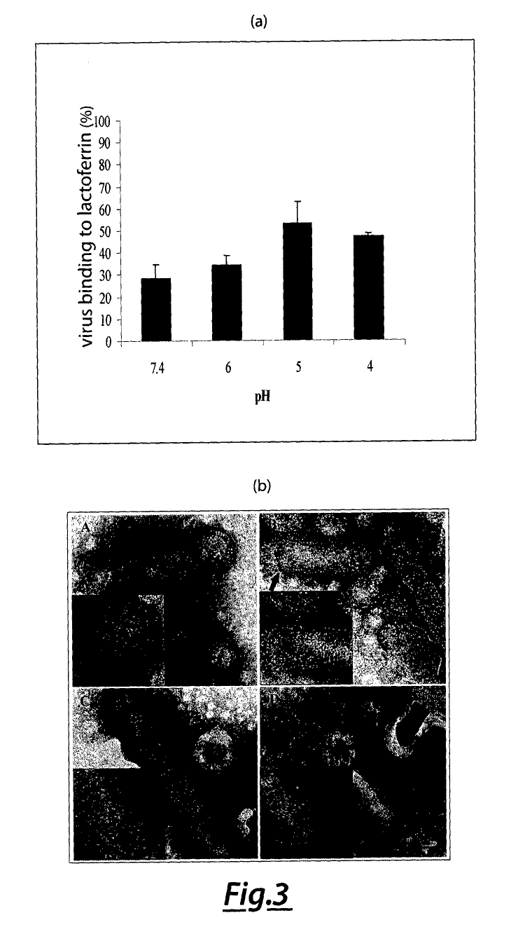

FIG. 3 shows (a) influence of pH on Influenza virus binding to bLf by Enzyme-linked immunosorbent assay (ELISA). Influenza virus A/RomaISS/2/08 was treated with different acidic buffers (pH 4.0, 5.0, 6.0, and 7.4) and allowed to bind to plastic-adsorbed bLf. Viral binding was detected by an ELISA by anti-influenza A antibody staining and results are expressed as percentage of virus binding to bLf. Data represents the means of at least three independent experiments. (b) Transmission Electron Microscopy of Influenza virus A/RomaISS/2/08 H1N1. (A) Influenza virus incubated at neutral pH. Note the spikes are well-ordered. (B) Virus incubated at pH 6.0. Spikes on the virions are still well defined and distinct. (C) Virus incubated at pH 5.0. Note that the spikes on the virions have become disorganized and are no longer well defined or distinct. (D) Virus incubated at pH 4.0. The spikes appear very disordered and discontinue.

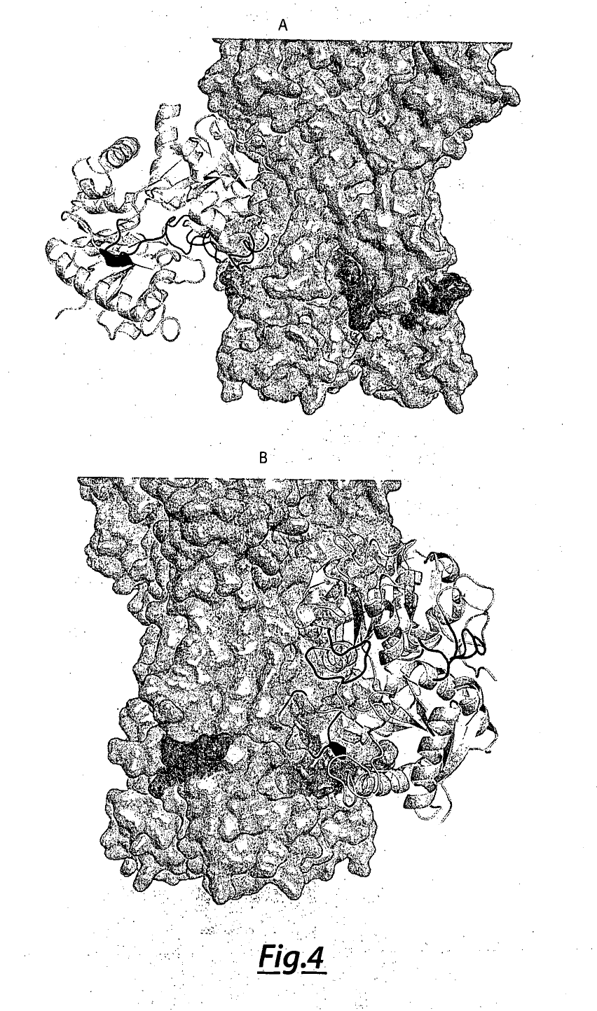

FIG. 4 shows putative interactions of bLf C-lobe (white ribbon) with HA stem (solid surface light grey): (A) in the cleft between two monomers; (B) close to the fusion peptide (dark grey). Main selected bLf sequences (SEQ ID NO: 1, 2 and 3) correspond to the black loops.

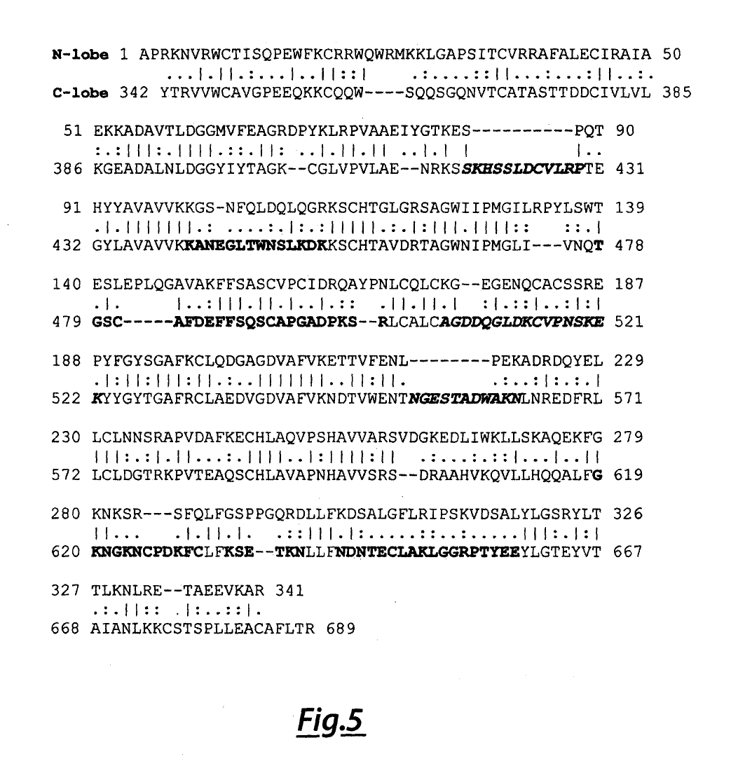

FIG. 5 shows sequence alignment of the N- (SEQ ID NO: 7) and C-lobe (SEQ ID NO:5) of bLf. The sequences in bold type correspond to loops supposed to be involved in binding. In bold italic main sequences are highlighted.

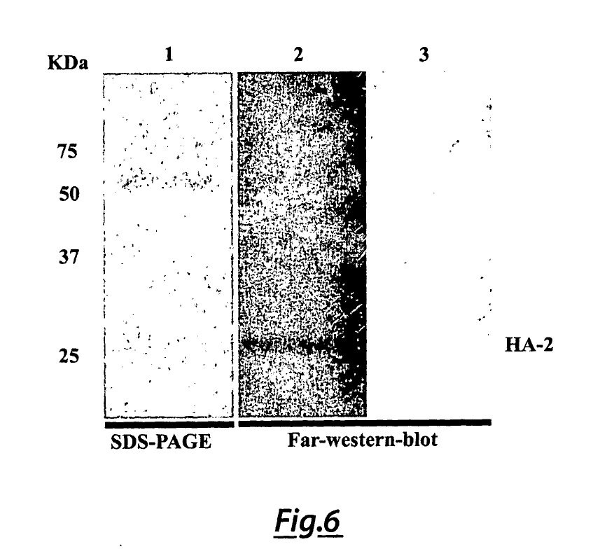

FIG. 6 shows ligand-blot assay of bLf binding to Influenza SW X-179A swine pandemic (H1N1) subunits. Lane 1: SDS-PAGE of reduced SW X-179A swine pandemic (H1N1) subunits. Lane 2: lactoferrin blot overlay. Lane 3: specificity control (rabbit anti-Lf antibodies+HRP-conjugated anti-rabbit antibodies).

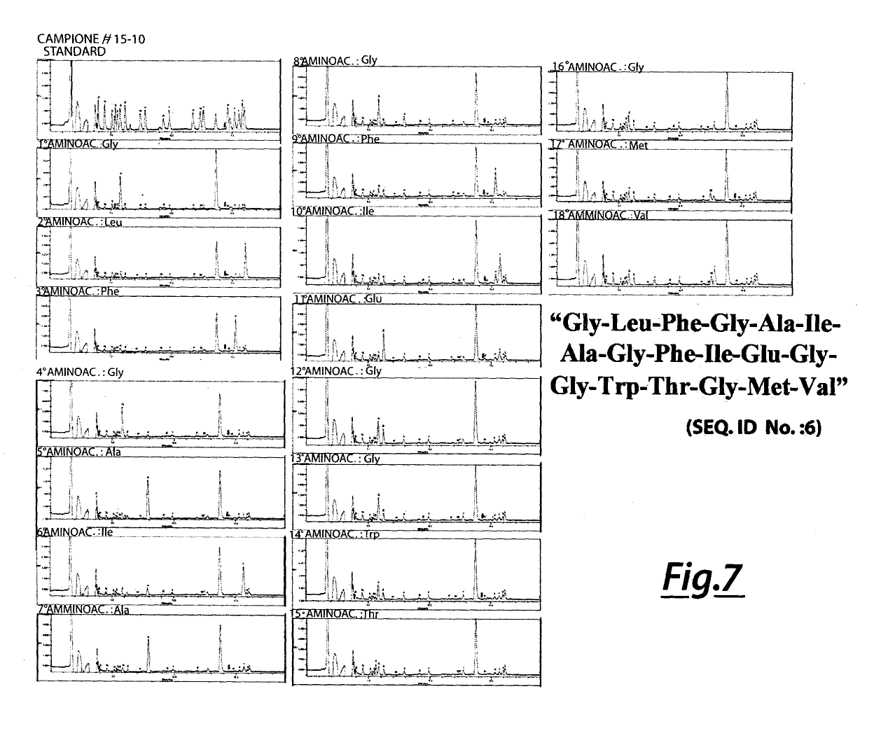

FIG. 7 shows N-terminal amino acid sequences determined by Edman degradation method.

### EXAMPLE 1

### Study on the Effectiveness of the Peptides of the Present Invention Against Influenza Virus Infection

Materials and Methods

Virus Strains

The following Influenza A virus strains were used: A/Solomon 3/2006 H1N1, A/Wisconsin/67/2005 H3N2, and SW X-179A swine pandemic H1N1 (inactivated vaccines and corresponding subunit preparations); A/Puerto Rico/8/34 H1N1 (PR8 virus), A/RomaISS/2/08 H1N1 oseltamivir-sensitive virus, A/Parma/24/09 H1N1 oseltamivir-resistant virus, A/Parma/05/06 H3N2, Avian 29/05/06 H5N1 (inactivated subunit vaccine), and A/Turkey/Italy/2676/99 H7N1 (kindly provided by Dr. Isabella Donatelli, ISS, Italy). PR8 virus was grown in the allantoic cavities of 10-day-old embryonated chicken eggs. After 48 h at 37° C., the allantoic fluid was harvested and centrifuged at 5000 rpm for 30 min to remove cellular debris, and virus titers were determined by a hemagglutinin titration and plaque assay according to the standard procedures (Gaush and Smith, 1968; Kurokawa et al. 1990). A/RomaISS/2/08 H1N1, A/Parma/24/09 H1N1, and A/Parma/05/06 H3N2 viruses were grown in Madin-Darby canine kidney (MDCK) cells. MDCK cells were grown at 37° C. in a humidified atmosphere with 5% CO₂ in Minimal Essential Medium (MEM, Gibco; Paisley, UK) containing 1.2 g/L NaHCO₃, and supplemented with 10% inactivated fetal calf serum (FCS, Flow Laboratories, Irvine, UK.), 2 mM glutamine, 2% non essential amino acids (Gibco; Paisley, UK), penicillin (100 IU/ml, and streptomycin (100 μg/ml). Viruses were inoculated onto confluent cell monolayers grown in roller bottles at a multiplicity of infection (m.o.i.) of 1 plaque forming units (p.f.u.)/cell. After 90 min at 35° C., the inoculum was removed and the monolayers were washed three times with Phosphate Buffered Saline (PBS, pH 7.4) and then incubated at 35° C. in culture medium containing 2% non essential amino acids (Gibco; Paisley, UK), 4% Bovine Serum Albumin (BSA fraction V, Gibco; Paisley, UK) and 0.5 μg of N-tosyl-L-phenylalanine chloromethyl ketone-treated trypsin (TPCK trypsin; Sigma Chemical Co.; St. Louis, Mo., USA). When extensive cytopathic effect (c.p.e.) was observed, infected cultures were frozen and thawed three times, centrifuged (3000 rpm, 10 min), and supernatants were stored at −80° C. The infectivity titer was determined by hemagglutinin titration and plaque assay, according to the standard procedures (Gaush and Smith, 1968; Kurokawa et al., 1990).

Virus Purification

For virus purification infected allantoic fluid or supernatants from infected cultures were harvested and centrifuged at 1000 g for 30 min to remove debris. The virus was pelleted at 25000 g for 20 h. The pellet was resuspended in 10 mM HEPES buffer saline (HBS) for 1 h at 4° C., homogenized, incubated at 20° C. for 45 min, and centrifuged at 2000 g for 15 min to remove aggregated virus. The supernatant was placed atop a 0/20/40/60% sucrose density step gradient and centrifuged at 85000 g for 2 h. The purified virus was collected from the 20/40% sucrose interface, assayed for protein, quickly frozen, and stored at −80° C.

Lactoferrin

Lactoferrin from bovine milk (bLf), purchased from Morinaga Milk Industries (Zama City, Japan), was deprived of endotoxin as previously described (Pietrantoni et al., 2006). Detoxified bLf was dissolved as stock solution (400 mM) in pyrogen-free PBS. BLf purity was checked by SDS-PAGE (Sodium Dodecyl Sulphate-PolyAcrylamide Gel Electrophoresis) stained with silver nitrate and was judged to be greater than 95%. Protein concentration was determined by UV spectroscopy on the basis of the extinction coefficient of 15.1 (280 nm, 1% solution) (Groves, 1960). The iron saturation rate of bLf, determined by atomic absorption spectrometry, was approximately 19.4%.

Enzymatic Hydrolysis of bLf and HPLC Separation, Purification and Characterisation of N- and C-Lobes

BLf (4 mg/ml) was dissolved in 50 mM ammonium bicarbonate, pH 8.5, and trypsin (from bovine pancreas, TPCK treated, Sigma Chemical Company, St. Louis, Mo., USA) digestion was performed at 37° C. overnight using an enzyme to substrate ratio of 1:50 w/w. The N- and C-lobes obtained by enzymatic hydrolysis were purified by reverse phase HPLC on a Vydac C18 column (250×10 mm, 5 μm) using a Waters HPLC System (Datasystem Millenium, HPLC pumps Waters 510, Detector Waters 486). Eluents were 0.1% trifluoroacetic acid (solvent A) and 0.07% trifluoroacetic acid in 95% acetonitrile (solvent B). The elution was performed by means of a short linear gradient from 35% to 55% solvent B over 20 min. at a flow rate of 3.5 ml/min. and monitored at 220 nm. The trypsin-containing fraction was discharged since it did not co-eluted with fractions containing bLf lobes. In addition, control experiments did not show residual tryptic activity in the digested fractions of bLf since the enzyme underwent auto-hydrolysis and inactivation due to denaturing conditions of HPLC. Electrophoretic analysis (SDS-PAGE) of the HPLC fractions has been carried out using 12.5% gels stained by Comassie Blue R250. Purification and characterisation of bLf N- and C-lobes were carried out as previously described (Superti et al., 2001). Fractions containing bLf lobes were collected, dried in a Speed-Vac centrifuge (Savant), lyophilised twice and stored at −20° C.

Peptide Synthesis

Peptide synthesis was performed in Solid Phase Peptide Synthesis (SPPS) with the Fmoc-chemistry by Primm Biotech, Inc., Milan, Italy. The peptide chains have been assembled on a solid support starting from the C-terminus and progressing towards the N-terminus, by repeating three basic steps: deprotection of the Fmoc (9-fluorenylmethyloxycarbonyl) alpha amino group of the amino acid, activation of the next protected amino acid, as an active ester, and coupling. After the desired sequence of amino acids was obtained, the peptide was removed from the polymeric support via TFA (Trifluoroacetic Acid) cleavage with concomitant removal of the protecting groups on the amino acid side-chains.

Cytotoxicity Assay

To establish the maximal non-cytotoxic dose of bLf and its derivatives, two-fold serial dilutions of each substance in culture medium were incubated at 37° C. with confluent MDCK cells grown in 96-well tissue culture microplates (Nalge Europe Ltd, Neerijse, Belgium). After 24 h, the following parameters were evaluated: cell morphology and viability (determined by neutral red staining) were examined by light microscopy and cell proliferation was evaluated quantitatively by microscopic counts after dispersion into individual cells with trypsin. Substance dilutions that did not affect any of these parameters were considered as non-cytotoxic concentrations and utilised for antiviral assays.

Inhibition of Hemagglutination

Hemagglutination assays were carried out in U-bottomed microtitre plates using 50 μl of 0.5% suspensions of turkey red blood cells (rbcs) in PBS added to 50 μl virus serially diluted in PBS. For testing bLf or peptidic fragments for inhibition of hemagglutination, virus in PBS was incubated for 60 min. at 4° C. with equal volume of serial dilutions of bLf or its peptidic fragments in PBS, starting from 12.5 μM. An equal volume of 0.5% turkey rbcs in PBS was then added and allowed to agglutinate. Titres were expressed as the reciprocal of the bLf or its peptidic fragment dilution giving 50% inhibition of hemagglutination by four agglutinating units of virus.

Neutralization Assay

Neutralization of virus binding to MDCK cells was carried out by incubating serial twofold bLf or peptidic fragment dilutions, starting from 12.5 μM, in culture medium with equal volumes (0.25 ml) of virus suspension containing 1.10⁶ p.f.u./0.25 ml. In negative controls, culture medium was used instead of bLf or peptidic fragments in the same volume. The mixtures were incubated for 60 min. at 4° C. MDCK cell monolayers, grown in 96 well tissue culture microplates (Nalge Europe Ltd, Neerijse, Belgium) for 24 h at 37° C. in 5% CO₂-air, were infected with 100 μl/well (in quadruplicate) of the virus-protein mixtures. It was noted that the virus/cell ratio was very important in this type of neutralization assay and the optimal ratio was to infect 20,000 cells with 2.10⁵ p.f.u. of virus in 96-well plates (m.o.i. 10). After 1 hour adsorption at 37° C., cells were rinsed thoroughly and incubated at 37° C. for 24 hours. The viral cytopathic effect (c.p.e.) was measured by neutral red staining. After staining and washes, the healthy MDCK cells took the neutral red dye but not the damaged cells after being infected by Influenza virus. The proportions of healthy cells have been determined by reading the absorbance of neutral red at A540 of each well. The fraction of viable cells in the presence of neutralizing substances was quantified by the staining of cells, where the intensity of the staining was directly proportional to the number of viable cells. Percent neutralization was determined by the formula below:

$$\% neutralization \text{:} \frac{\begin{matrix}{A}_{540} of substance testing wells - \\ {A}_{540} of viral control wells\end{matrix}}{\begin{matrix}{A}_{540} of cell control wells - \\ {A}_{540} of viral control wells\end{matrix}} \times 100$$

The neutralizing substance titer in neutral red assay has been reported as IC₅₀, the reciprocal substance dilution at which 50% of MDCK cells were protected from the virus induced killing.

Neutral Red Uptake Assay

For neutral red uptake assay, treated and untreated infected cells were stained for 3 h with neutral red (50 μg/ml, 200 μl/well, 37° C., 5% CO₂); then the cells were washed with Hank's salt solution and fixed for 10 min. at room temperature with 4% formaldehyde, 10% CaCl₂ (200 μl/well). The uptaken dye was extracted for 15 min. at room temperature by 1% acetic acid in 50% ethanol (200 μl/well) and the damage of the cells by the virus or the possible protection by the compounds were measured at 540 nm in an ELISA-reader.

Far-Western-Blot

Purified viral subunits preparations of Solomon (H1N1) and Wisconsin (H3N2) strains were resolved by SDS-PAGE, as described by Laemmli (1970) under denaturing or/and non-denaturing conditions on 15% acrylamide gel. Proteins were then transferred from gel to nitrocellulose membranes (Bio-Rad, Hercules, Calif., USA), using a Semidry Transfer Cell apparatus (Trans-blot, Bio-Rad, Hercules, Calif., USA) according to Manufacturer's protocol. After transfer, the membranes were blocked with 5% skim-milk solution for 1 h, followed by washing with PBS/0.05% Tween-20 (washing solution). Membranes were then incubated for 90 min. with washing solution containing 1 mg/ml (12.5 μM) bLf. After extensive washing, membranes were incubated with rabbit anti-lactoferrin antibodies (Sigma Chemical Company, St. Louis, Mo., USA) in a washing solution for 1 h, washed again, and then incubated 1 h with goat horseradish peroxidase (HRP)-labelled anti-rabbit IgG antibodies (Bio-Rad, Hercules, Calif., USA). Following extensive washing, staining was achieved by using 3,3′,5,5′-tetramethylbenzidine (TMB) substrate kit for peroxidase (Vector Laboratories, Inc., Burlingame, Calif., USA) according to Manufacturer's instructions. All incubations were carried out at room temperature. Non-specific anti-Lf antibody binding to viral proteins was verified by incubating nitrocellulose strips with rabbit anti-Lf antibodies followed by HRP-conjugated anti-rabbit antibodies.

Sequencing

For identification of HA region recognized from bLf in far-western blot, A/Solomon 3/2006 H1N1 subunits were separated on 15% acrylamide gel in denaturing conditions. Separated proteins were then transferred from gel to polyvinylidene difluoride (PVDF) membrane (Bio-Rad, Hercules, Calif., USA), using a Semidry Transfer Cell apparatus (Trans-blot, Bio-Rad, Hercules, Calif., USA) according to Manufacturer's protocol. Transfer was performed using CAPS [3-Cyclohexylamino-1-propanesulfonic acid] 10 mM buffer (pH 11.0) containing 10% methanol. Membrane was then stained by Comassie-Blue R-250 solution (0.25% w/v) in 50% methanol for about 5 min., washed with water and destained in 50% methanol with constant shaking. The protein band of interest was excised, extensively washed with sterile Milli-Q water, and the N-terminal amino acid sequences were determined by Edman degradation method by Primm Biotech, Inc., Milan, Italy.

Enzyme-Linked Immunosorbent Assay (ELISA)

ELISA was performed to determine lactoferrin binding to viral particles treated at appropriate pH. The A/RomaISS/2/08 and A/Parma/24/09 viruses were treated with THE buffer (0.1 M Tris, 1 M NaCl, 0.05 M Na EDTA) at different pH (pH 7.4, 6.0, 5.0, and 4.0). After incubation at 37° C. for 15 min., the reaction was neutralized with NaOH and viruses used for binding assay. Flat-bottomed 96-well plates (Nalge Europe Ltd., Neerijse, Belgium) were coated with 0.1 mg/well of lactoferrin in 0.05 M carbonate buffer (pH 9.6) at 4° C. overnight. The plates were blocked with 10% bovine serum albumin (BSA) in PBS for 2 h at 37° C. After washing with PBS containing 0.01% Tween-20 (PBS-T), the plates were incubated at 37° C. for 1 h with 50 μl of purified viral particles pre-treated for 15 min at 37° C. with PBS at different pH (pH 7.4, 6.0, 5.0, and 4.0). The plates were washed and incubated at 37° C. for 1 h with chicken anti-influenza A antibodies (Abcam plc, Cambridge, UK) in PBS containing 1% BSA, washed again and incubated at 37° C. for 1 h with HRP-conjugated polyclonal rabbit anti-chicken IgG (Sigma Chemical Co.; St. Louis, Mo., USA). After washing with PBS-T, the peroxidase substrate o-phenylendiamine (OPD) dihydrochloride was added. The developing of colour reaction was stopped by adding 50 μL of 3.0 N HCl. The optical density was read on a multilabel plate reader (PerkinElmer Italia, Monza) at 490 nm.

Transmission Electron Microscopy (TEM)

For TEM visualization, purified viral particles were treated for 15 min at 37° C. with PBS at different pH (pH 7.4, 6.0, 5.0, and 4.0). After pH neutralization, 20 μl of each sample were absorbed onto carbon-formvar-coated, 400-mesh copper grids, stained with 2% phosphotungstic acid (PTA) (pH 7.0) for 30 s, and observed on a Philips 208S electron microscope at 80 kV.

Computational Methods

The HA peptide sequences were obtained for the virus strains A/Solomon 3/2006 H1N1, A/Wisconsin/67/2005 H3N2, A/Puerto Rico/8/34 H1N1 (PR8 virus), A/RomaISS/2/08 H1N1 and A/Parma/24/09 H1N1. The above sequences were aligned to those of the HA whose X-ray structures were available in the Protein Data Bank. The BlastX program was used to translate the nucleotide sequence in aminoacid sequence. FASTA sequences alignment was carried out applying the following parameters: algorithm: EMBOSS, method: needle, matrix: Blosum62, gap open penalty: −10; gap extend penalty: −0.5. The same procedure was applied to align the N- and C-lobe of bLf obtained by cutting the complete sequence at the residue number 342.

The protein-protein docking calculations were performed using the X-ray structures of HA with PDB code 2WRG and of the bLf C-lobe of with PDB code 3IB0. The two crystal structures were submitted to the Protein Preparation routine in Maestro (Schrodinger, N.Y., USA) that allows fixing up receptor structure, eliminating water and sugar molecules, fixing bond order, adding hydrogen atoms, ionising lysine, arginine, glutamate and aspartate residues. To optimize the hydrogen bond network, histidine tautomers and ionization states were predicted, 180° rotations of the terminal χ angle of Asn, Gln, and His residues were assigned, and hydroxyl and thiol hydrogen atoms were sampled. For each structure, a brief relaxation was performed using an all-atom constrained minimization carried out with the Impact Refinement module (Impact, Schrodinger, N.Y., USA) using the OPLS-2005 force field, to reduce steric clashes that may exist in the original PDB structures. The minimization was terminated when the energy converged or the RMSD reached a maximum cutoff of 0.30 Å. The 3IB0 protein was preliminarily passed through the Prime (Prime, Schrodinger, N.Y., USA) homology modelling routine to complete the structure with missing residues.

So prepared structures were submitted to different docking procedures using four softwares (Autodock-MacroModel, ZDock-RDock, PatchDock-FireDock and ClusPro). All of them comprise a preliminary docking calculation followed by a refinement step.

1. A rigid docking calculation was performed with Autodock, setting the grid on the stem region of just one HA monomer. Docking calculations were carried out employing the Lamarckian Genetic Algorithm as research algorithm implemented in Autodock. The following settings were used: number of grid points in xyz 126 126 126, spacing 1.0 Å, number of the individuals in population 300, maximum number of energy evaluations 2500000, maximum number of generations 30000, hybrid GA-LS, runs 250. After ordering all the conformations by docked energy, the docking poses were clustered by means of ADTools, applying an RMSD cutoff of 0.5 Å. Most representative complexes of each cluster were subsequently fully minimized (with no constraints) with MacroModel (MacroModel, Schrodinger, N.Y., USA) applying the following settings: force field OPLS-2005, solvent Water, maximum number of iterations: 10000, minimization method PRCG, convergence on gradient with a threshold of 0.05.

2. ZDock and RDock are implemented in Discovery Studio (Discovery Studio, Accelrys, San Diego, USA). Docking calculations were performed setting the following parameters: residues of the upper part of HA and residues of C-lobe of bLf in close contact with the N-lobe were blocked (not explored), 54000 initial docked poses were generated, and the best 2000 poses were ranked by ZRank and then clustered to obtain 100 clusters (RMSD>10 Å). Most representative complexes of each cluster were submitted to following refinement with RDock applying the default parameters.

3. PatchDock is a web server (http://bioinfo3d.cs.tau.ac.il/PatchDock) that performs docking calculation using an algorithm based on shape complementarity principles (rigid-body). Default parameters were applied and 100 complexes (clustered with an RMSD cutoff of 4.0 Å) were passed to subsequent refinement with FireDock. This latter is a web server that performs large scale flexible refinement, by restricted interface side-chain rearrangement and by soft rigid-body optimization.

4. ClusPro is a web server (http://cluspro.bu.edu/home.php) that exploits PIPER, an FFT-based docking algorithm, to generate 1000 complexes, that were subsequently clustered. Best cluster representatives were submitted to refinement via Monte Carlo simulation and applying the Semi-Defined programming based Underestimation (SDU) to score the complexes.

All complexes resulting from the four docking calculations were collected and analysed in order to remove unsuitable poses.

The selected complexes were clustered in order to find a consensus between poses generated with different approaches. The clustering was carried out using R (R, The R foundation for statistical computing) and applying the Ward clustering metric.

161 resulting complexes were aligned to each other and visually inspected using PyMOL v 1.2 (PyMOL, DeLano Scientific, San Francisco, USA).

Results

Interaction of bLf with Viral Hemagglutinin

The first step of Influenza virus entry into susceptible cells depends on the interaction between the viral HA and a specific sialic acid-containing cell receptor. As receptor binding of functional HA can be measured by agglutination of turkey erythrocytes, direct interaction between virus and bLf was tested by checking the inhibition of viral hemagglutination activity. As shown in Table 1 (Interaction of bLf with viral HA), concentrations of bLf ranging from about 0.05 pM to 6 nM were able to prevent hemagglutination activity of all tested virus strains. Neither bLf-induced agglutination nor visual evidence of erythrocyte lysis was observed in the above assays, thus bLf inhibition of viral hemagglutination was considered genuine.

TABLE 1

| Viral strain | subtype | HI titre | |
|---|---|---|---|
| A/Roma-ISS/2/08 (Brisbane-like) (oseltamivir- | H1N1 | 6 | nM |
| sensitive) | | | |
| A/Parma/24/09 (Brisbane-like) (oseltamivir- | H1N1 | 6 | nM |
| resistant) | | | |
| A/PR/8/34 | H1N1 | 3 | nM |
| A/Solomon (3/06) inactivated subunits, vaccine | H1N1 | 5 | pM |
| A/Parma/5/06 (Wisconsin-like) | H3N2 | 3 | nM |
| A/Wisconsin (67/05) inactivated subunits, | H3N2 | 0.3 | pM |
| vaccine | | | |
| SW X-179A 1089/09 inactivated subunits, | H1N1 | 46.5 | fM |
| vaccine | | | |
| Avian 29/05/06 inactivated vaccine | H5N1 | 2.9 | pM |
| A/Turkey/Italy/2676/99 | H7N1 | 93.1 | fM |

Neutralization of Influenza Virus Infection by Lactoferrin

It has been examined whether, and to what extent, bLf could affect virus replication. This was tested by neutral red staining measuring of virus-infected MDCK cells as a measure of cell viability. Two strains of the Influenza A/H1N1 virus subtype (A/Roma-ISS/2/08 and A/Parma/24/09), and one strain of A/H3N2 virus subtype (A/Parma/05/06) were used in these experiments. Virus replication was markedly inhibited by bLf, at concentrations several logs lower than those causing non-specific MDCK cytotoxicity. In order to determine the selectivity index (SI) of lactoferrin, the ratio between the 50% drug cytotoxicity concentration (CC₅₀) and the concentration required to inhibit the viral cytopathic effect by 50% (EC₅₀) was calculated. SI values were between about 10⁶ and 10⁵ for Influenza A/H1N1 and A/H3N2 virus subtypes, respectively (data not shown).

Far-Western-Blot and Protein Identification

Based on the data reported above, we attempted to identify the actual bLf-virus binding components, and started by examining bLf interaction with virus envelope proteins. To this aim, HA and NA surface antigens present in Solomon (H1N1) and Wisconsin (H3N2) subunit vaccines were separated by electrophoresis and probed with bLf by a far-western blot assay. FIGS. 1 and 2 show that in both viral strains bLf more consistently binds to a viral protein of apparent molecular mass of 28 kDa, tentatively corresponding to the HA₂ subunit of HA protein.

Similar results have been obtained with subunits vaccine of SW X-179A swine pandemic (H1N1) strain (FIG. 6).

N-terminus protein sequencing of H1N1 Solomon subunit sample confirmed that the virus component bound by bLf was indeed the HA₂ subunit of HA. In particular, the first 18 aminoacids of N-terminus sequence were GLFGAIAGFIEGGWTGMV (SEQ ID No: 6) corresponding to the highly conserved fusion peptide of HA₂ subunit (Ekiert et al., 2009) (FIG. 7).

pH Assay

Since bLf recognizes HA₂ subunit, which is involved in pH-induced conformational changes during Influenza virus entry into the cells of the susceptible host, experiments were carried out to determine lactoferrin binding to viral particles exposed to different pH values. These experiments were performed, with similar result, with two viral strains, but the data in FIG. 3 refer to the Influenza A/RomaISS/2/08 viral strain. BLf was found to bind to the virus with the highest affinity at acidic pH (52.9+/−9.7% and 34.1+/−4.1% at pH 5.0 and 6.0, respectively). BLf binding strength was still high at pH 4.0 (47.2+/−1.4%) (FIG. 3a). When Influenza virus was observed by transmission electron microscopy after negative staining with phosphotungstic acid, untreated and acid-treated virus particles markedly differed in their morphology (FIG. 3b).

As shown in FIG. 3b, at neutral pH, the HA spikes were well-ordered, rectangular structures projecting from the virus membrane (A). Viral particles treated at pH 6.0 showed spikes on the virions still distinct and well defined (B). After incubation at pH 5.0, the well-ordered appearance was lost, and individual spikes were difficult to discern (C). This pattern was more evident after pH 4.0 treatment, that induced HA spikes to appear very disordered and barely distinguishable (D).

The loss of well-defined spike structure observed at low pH reflects the irreversible conformational change in HA (Ruigrok et al., 1986).

Effect of N-Lobe and C-Lobe on Viral Hemagglutination

BLf is made of two lobes, named N-lobe and C-lobe, respectively pertaining to the N-terminus and C-terminus moieties of the molecule. We therefore asked which of the two lobes was actually interacting with the HA₂ component of the Influenza virus, hence inhibiting viral hemagglutination. Results obtained are reported in Table 2 that shows the activity of N- and C-lobes on viral hemagglutination.

TABLE 2

| | | | C-lobe | | C-lobe | |
|---|---|---|---|---|---|---|
| | | N-lobe | (aa | | (aa | |
| Viral strain | Subtype | (aa 1-280) | 285-689) | | 345-689) | |
| A/Roma-ISS/2/08 | H1N1 | — | 10 | nM | 10 | nM |
| A/Parma/24/09 | H1N1 | — | 24 | nM | 10 | nM |
| A/PR/8/34 | H1N1 | — | 24 | nM | 12 | nM |
| A/Solomon (3/06) | H1N1 | — | 5 | pM | 5 | pM |
| inactivated SU vaccine | | | | | | |
| A/Parma/5/06 (Wisconsin- | H3N2 | — | 6 | nM | 6 | nM |
| like) | | | | | | |
| A/Wisconsin (67/05) | H3N2 | — | 1.6 | pM | 1.2 | pM |
| inactivated SU vaccine | | | | | | |
| SW X-179A 1089/09 | H1N1 | — | 46.5 | fM | 46.5 | fM |
| inactivated SU vaccine | | | | | | |
| Avian 29/05/06 inactivated | H5N1 | — | 2.9 | pM | 2.4 | pM |
| vaccine | | | | | | |
| A/Turkey/Italy/2676/99 | H7N1 | — | 93.1 | fM | 93.1 | fM |

A marked inhibition of viral hemagglutination was obtained with C-lobe whereas N-lobe showed no inhibitory activity.

Neutralization of Influenza Virus Infection by N- and C-Lobes

We next examined whether, and to what extent, N- and C-lobes of bLf could affect virus replication. Two strains of the Influenza A/H1N1 virus subtype (A/Roma-ISS/2/08 and A/Parma/24/09), and one strain of A/H3N2 virus subtype (A/Parma/05/06) were used in these experiments. As expected, virus infection was not affected by N-lobe treatment whereas replication was highly inhibited by C-lobe. SI values were 2.5-5 fold higher than bLf (data not shown).

Protein-Protein Docking

The data above show that bLf interacts with HA and suggest a possible interaction between the C-lobe of bLf and the fusion peptide (HA₂ subunit) of HA. In order to gain more insight into the binding mode between bLf and HA a protein-protein docking protocol has been setup because of the lack of structural data (X-ray or NMR) about the interaction between these two proteins.

For the docking calculations the X-ray structure of the C-lobe of bLf with PDB code 3IB0 was used, as this portion is responsible for the bLf activity (see above). The crystal structure of HA with PDB code 2WRG was selected because it showed the highest sequence similarity with the HAs isolated from the viral strain of H1N1 used in the assays. Table 3 shows the percentage of sequence identity and similarity of indicated strains hemagglutinins with the crystal structure 2WRG.

TABLE 3

| Viral strain | subype | % Identity^{a} | % Similarity^{a} |
|---|---|---|---|
| A/Solomon (3/06) | H1N1 | 82 | 88 |
| inactivated subunits, | | | |
| vaccine | | | |
| A/Roma-ISS/2/08 | H1N1 | 84 | 90 |
| A/Parma/24/09 | H1N1 | 84 | 89 |
| A/PR/8/34 | H1N1 | 86 | 90 |

^{a}values calculated with the Multiple Sequence Viewer of Maestro.

Noteworthy, major differences were found in the residues of the HA₁ subunit, whereas the HA₂ sequence was perfectly conserved among all of the considered HAs belonging to the H1N1 strains, demonstrating that this region is an interesting target for anti-Influenza therapies.

Docking calculations were performed using four different software (ZDOCK, PatchDock, AutoDock, ClusPro) selected as they use different approaches. Results obtained from all procedures were analysed with the aim to find a consensus: finding common binding modes applying diverse docking protocols can improve the reliability of the observed results; it is well known that protein-protein docking is a challenging computational approach that provide many results because of the large dimensions of the interacting protein surfaces.

All the simulations were carried out using the C-lobe of bLf to speed up calculation, as experimental data showed that N-lobe was inactive. Moreover, as the Far-Western-blot analysis demonstrated the binding at the HA₂ subunit, binding poses involving this region of HA were selected for following analysis.

The results were analysed to eliminate all the unsuitable binding poses, involving not accessible regions of bLf or HA.

As shown in FIG. 4, most of the binding poses concentrated on two regions of the HA stem: the first one in the cleft between two monomers (FIG. 4A) and the second one very close to the fusion peptide (FIG. 4B). The latter binding site seems of particular interest as it corresponds to the region targeted from universal antibodies. Therefore binding to this region could explain why bLf inhibition is exerted toward all the viral strains under analysis.

C-lobe of bLf can bind in many different ways to HA: interaction to hemagglutinin is most frequently mediated by three loops: SKHSSLDCVLRP (aa 418-429)(SEQ ID NO:1), NGESTADWAKN, (aa 553-563) (SEQ ID NO:3) and AGDDQGLDKCVPNSKEK (aa 506-522) (SEQ ID NO:2). However also other sequences corresponding to solvent exposed, accessible loops of bLf can contribute to the overall interaction with HA: KANEGLTWNSLKDK (441-454) (SEQ ID NO: 8), TGSCAFDEFFSQSCAPGADPKSR (478-501) (SEQ ID NO: 9), GKNGKNCPDKFC (619-630) (SEQ ID NO: 10), KSETKN (633-637) (SEQ ID NO: 11), NDNTECLAKLGGRPTYEE (642-659) (SEQ ID NO: 12) Alignment of the sequences of the C- and the inactive N-lobe (FIG. 5) demonstrates that sequences 418-429 and 553-563 correspond to loops that are not present in the N-lobe, while the other selected loops show a sequence identity with respect to N-lobe<33% (see Table 4).

Because of poor stability of NGESTADWAKN peptide, a modified sequence has been designed where the threonine residue is substituted by a homologue serine, i.e. NGESSADWAKN (SEQ ID NO: 4). The peptides with SEQ ID NO: 1, 2 and 4 were tested in Hl and Nt assays.

TABLE 4

| SEQ | | | Identity |
|---|---|---|---|
| ID | | | vs N- |
| NO | Residues | Sequence | lobe (%) |
| 1 | 418-429 | SKHSSLDCVLRP | 8 |
| | | (SEQ ID NO: 1) | |
| 2 | 506-522 | AGDDQGLDKCVPNSKEK | 29 |
| | | (SEQ ID NO: 2) | |
| 3 | 553-563 | NGESTADWAKN | 0 |
| | | (SEQ ID NO: 3) | |
| 4 | — | NGESSADWAKN | — |
| | | (SEQ ID NO: 4) | |
| 8 | 441-454 | KANEGLTWNSLKDK | 14 |
| | | (SEQ ID NO: 8) | |
| 9 | 478-501 | TGSCAFDEFFSQSCAPGADPKSR | 33 |
| | | (SEQ ID NO: 9) | |
| 10 | 619-630 | GKNGKNCPDKFC | 33 |
| | | (SEQ ID NO: 10) | |
| 11 | 633-637 | KSETKN | 12 |
| | | (SEQ ID NO: 11) | |
| 12 | 642-659 | NDNTECLAKLGGRPTYEE | 6 |
| | | (SEQ ID NO: 12) | |

Interaction of bLf Derived Peptides with Viral Hemagglutinin

The direct interaction between virus and bLf-derived peptides was tested by checking the inhibition of viral hemagglutination. As shown in Table 4, SKHSSLDCVLRP (amino acid residues 418-429) (SEQ ID NO:1, AGDDQGLDKCVPNSKEK (amino acid residues 506-522) (SEQ ID NO:2), and NGESSADWAKN (SEQ ID NO: 4) were able to prevent hemagglutinating activity of all tested virus strains at higher extent compared to the entire protein.

TABLE 5

| | | SEQ ID | | SEQ ID | | SEQ ID | |
|---|---|---|---|---|---|---|---|
| | | No: 1 | | NO: 2 | | No: 4 | |
| Viral strain | subtype | HI titre | | HI titre | | HI titre | |
| A/Roma-ISS/2/08 | H1N1 | 1.4 | pM | 1.4 | pM | 0.7 | fM |
| A/Parma/24/09 | H1N1 | 1.4 | pM | 1.4 | pM | 0.3 | fM |
| A/PR/8/34 | H1N1 | 0.7 | nM | 0.35 | nM | 0.7 | fM |
| A/Solomon (3/06) | H1N1 | 46.5 | fM | 46.5 | fM | 93 | fM |
| inactivated subunits, | | | | | | | |
| vaccine | | | | | | | |
| A/Parma/5/06 (Wisconsin- | H3N2 | 0.7 | pM | 0.7 | pM | 0.3 | pM |
| like) | | | | | | | |
| A/Wisconsin (67/05) | H3N2 | 23.2 | fM | 11.6 | fM | 5.8 | fM |
| inactivated subunits, | | | | | | | |
| vaccine | | | | | | | |
| SW X-179A 1089/09 | H1N1 | 5.8 | fM | 46 | fM | 0.3 | fM |
| inactivated SU vaccine | | | | | | | |
| Avian 29/05/06 inactivated | H5N1 | 18 | fM | 70 | fM | 93.1 | fM |
| vaccine | | | | | | | |
| A/Turkey/Italy/2676/99 | H7N1 | 18 | fM | 46.5 | fM | 5.8 | fM |

As observed for bLf, neither peptide-induced agglutination nor visual evidence of erythrocyte lysis was detected in the above assays, thus also peptide inhibition of viral hemagglutination was considered genuine.

Neutralization of Influenza Virus Infection in MDCK Cells by bLf-Derived Peptides

We examined whether, and to what extent, bLf-derived peptides were able to affect virus replication. Two strains of the Influenza A/H1N1 virus subtype (A/Roma-ISS/2/08 and A/Parma/24/09), and one strain of A/H3N2 virus subtype (A/Parma/05/06) were used in these experiments. As shown in Tables 6, 7, and 8, bLf-derived peptides were better inhibitors than the entire protein, their selectivity index being about one or two order of magnitude higher, depending on virus strain. Concerning NGESSADWAKN (SEQ ID NO:4), the SI in H1N1 (oseltamivir resistant strain) infected cells reaches values 500 times higher than bLf.

TABLE 6
In vitro antiviral activity of SKHSSLDCVLRP
(SEQ ID NO: 1) towards Influenza
virus infection

| Viral strain | subype | CC₅₀ * | EC₅₀ ° | SI\hat{ } |
|---|---|---|---|---|
| A/Roma-ISS/2/08 | H1N1 | >25 μM | 4 pM | >6.25 · 10⁶ |
| A/Parma/24/09 | H1N1 | >25 μM | 3.1 pM | >8 · 10⁶ |
| A/Parma/05/06 | H3N2 | >25 μM | 5.8 pM | >4.3 · 10⁶ |

TABLE 7
In vitro antiviral activity of
AGDDQGLDKCVPNSKEK (SEQ ID NO: 2) towards
Influenza virus infection

| Viral strain | subype | CC₅₀ * | EC₅₀ ° | SI\hat{ } |
|---|---|---|---|---|
| A/Roma-ISS/2/08 | H1N1 | >25 μM | 3.7 pM | >6.75 · 10⁶ |
| A/Parma/24/09 | H1N1 | >25 μM | 3.4 pM | >7.35 · 10⁶ |
| A/Parma/05/06 | H3N2 | >25 μM | 7.3 pM | >3.42 · 10⁶ |

TABLE 8
In vitro antiviral activity of NGESSADWAKN
(SEQ ID NO: 4) towards Influenza
virus infection

| Viral strain | subtype | CC₅₀ * | EC₅₀ ° | SI\hat{ } |
|---|---|---|---|---|
| A/Roma- | H1N1 | >25 μM | 225 fM | >1.11 · 10⁸ |
| ISS/2/08 | | | | |
| A/Parma/24/09 | H1N1 | >25 μM | 50 fM | >5 · 10⁸ |
| A/Parma/05/06 | H3N2 | >25 μM | 22.5 pM | >1.11 · 10⁶ |

* CC₅₀ cytotoxic concentration 50%,
° EC₅₀ effective concentration 50%,
\hat{ }SI (selectivity index) = CC₅₀/EC₅₀

### REFERENCES

Ammendolia M G, Marchetti M, Superti F. (2007a) Bovine lactoferrin prevents the entry and intercellular spread of herpes simplex virus type 1 in Green Monkey Kidney cells. Antiviral Res. 76:252-262.
Ammendolia M G, Pietrantoni A, Tinari A, Valenti P, Superti F. 2007b. Bovine lactoferrin inhibits echovirus endocytic pathway by interacting with viral structural polypeptides. Antiviral Res. 73:151-60.
Andersen J H, Osbakk S A, Vorland L H, Traavik T, and Gutteberg T J. 2001. Lactoferrin and cyclic lactoferricin inhibit the entry of human cytomegalovirus into human fibroblasts. Antiviral Res. 51:141-149.
Anderson B F, Baker H M, Dodson E J, Norris G E, Rumball S V, Waters J M, Baker E N. 1987. Structure of human lactoferrin at 3.2-A resolution. Proc. Natl. Acad. Sci. USA 84:1769-1773.
Berkhout B, van Wamel J L, Beljaars L, Meijer D K, Visser S, Floris R. 2002. Characterization of the anti-HIV effects of native lactoferrin and other milk proteins and protein-derived peptides. Antiviral Res. 55:341-355.
Berkhout B, Floris R, Recio I, Visser S. 2004. The antiviral activity of the milk protein lactoferrin against the human immunodeficiency virus type 1. Biometals 17:291-294.
Cheng P K, To A P, Leung T W, Leung P C, Lee C W, Lim W W. 2010. Oseltamivir- and amantadine-resistant influenza virus A (H1N1), Emerg. Infect. Dis. 16:155-156.
Dharan N J, Gubareva L V, Meyer J J, Okomo-Adhiambo M, McClinton R C, Marshall S A, St George K, Epperson S, Brammer L, Klimov A I, Bresee J S, Fry A M, Oseltamivir-Resistance Working Group. 2009. Infections with oseltamivir-resistant influenza A(H1N1) virus in the United States. JAMA 30:1034-1041.
Di Biase A M, Pietrantoni A, Tinari A, Siciliano R, Valenti P, Antonimi G, Seganti L, Superti F. 2003. Heparin-interacting sites of bovine lactoferrin are involved in anti-adenovirus activity. J. Med. Virol. 69:495-502.
Dolin R, Reichman R C, Madore H P, Maynard R, Linton P N, Webber-Jones J. 1982. A controlled trial of amantadine and rimantadine in the prophylaxis of influenza A infection. N. Engl. J. Med. 307:580-584.
Edwards M, Dimmock N J. 2001. Hemagglutinin 1-specific immunoglobulin G and Fab molecules mediate postattachment neutralization of influenza A virus by inhibition of an early fusion event J. Virol. 75:10208-10218.
Ekiert D C, Bhabha G, Elsliger M A, Friesen R H, Jongeneelen M, Throsby M, Goudsmit J, Wilson I A. 2009. Antibody recognition of a highly conserved influenza virus epitope. Science 324:246-251
Fiore A E, Shay D K, Broder K, Iskander J K, Uyeki T M, Mootrey G, Bresee J S, Cox N S. Centers for Disease Control and Prevention (CDC). Advisory Committee on Immunization Practices (ACIP) 2008. Prevention and control of influenza: recommendations of the Advisory Committee on Immunization Practices (ACIP). MMWR Recomm. Rep. 57(RR-7):1-60.
Gaush C R, Smith T F. 1968. Replication and plaque assay of influenza virus in an established line of canine kidney cells. Appl. Microbiol. 16:588-594.
Groves M L 1960. The isolation of a red protein from milk. J. Am. Chem. Soc. 82:3345-3350.
Hara K, Ikeda M, Saito S, Matsumoto S, Numata K, Kato N, Tanaka K, Sekihara H. 2002. Lactoferrin inhibits hepatitis B virus infection in cultured human hepatocytes. Hepatol. Res. 24:228-235.
Harmsen M C, Swart P J, de B'ethune M P, Pawels R, De Clercq E, Th'e T H, Meijer D K F. 1995. Antiviral effects of plasma and milk proteins: lactoferrin shows a potent activity against both human immunodeficiency virus and human cytomegalovirus replication in vitro. J. Infect. Dis. 172:380-388
Hayden F G 2006. Antiviral resistance in influenza viruses—implications for management and pandemic response. N. Engl. J. Med. 354:785-788.
Kiso M, Mitamura K, Sakai-Tagawa Y, Shiraishi K, Kawakami C, Kimura K, Hayden F G, Sugaya N, Kawaoka Y. 2004. Resistant influenza A viruses in children treated with oseltamivir: descriptive study. Lancet 364:759-765.
Kurokawa M, Ochiai H, Nakajima K, Niwayama S. 1990. Inhibitory effect of protein kinase C inhibitor on the replication of influenza type A virus. J. Gen. Virol. 71:2149-2155.
Ikeda M, Nozaki A, Sugiyama K, Tanaka T, Naganuma A, Tanaka K, Sekihara H, Shimotohno K, Saito M, Kato N. 2000. Characterization of antiviral activity of lactoferrin against hepatitis C virus infection in human cultured cells. Virus Res. 66:51-63.
Ilyushina N A, Bovin N V, Webster R G, Govorkova, E A. 2006. Combination chemotherapy, a potential strategy for reducing the emergence of drug-resistant influenza A variants. Antiviral Res. 70: 121-131.
Laemmli U K 1970. Cleavage of structural proteins during the assembly of the head of bacteriophage T4. Nature 227:680-685.
Levay P F, Viljoen M. 1995. Lactoferrin: a general review. Haematologica 80:252-267.
Marchetti, Longhi C, Conte M P, Pisani S, Valenti P, Seganti L. 1996. Lactoferrin inhibits herpes simplex virus type 1 adsorption to Vero cells, Antiviral Res. 29:221-231.
Marchetti M, Superti F, Ammendolia M G, Rossi P, Valenti P, Seganti L. 1999. Inhibition of poliovirus type 1 infection by iron-, manganese and zinc-saturated lactoferrin. Med. Microbiol. Immunol. 187:199-204.
Marchetti M, Trybala E, Superti F, Johansson M, Bergstrom T. 2004. Inhibition of herpes simplex virus infection by lactoferrin is dependent on interference with the virus binding to glycosaminoglycans. Virology 18:405-413.
Marchetti M, Ammendolia M G, Superti F 2009. Glycosaminoglycans are not indispensable for the anti-herpes simplex virus type 2 activity of lactoferrin. Biochimie 91:155-159.
May J T. 1988. Microbial contaminants and antimicrobial properties of human milk. Microbiol. Sci. 5:42-46.
Murphy M E, Kariwa H, Mizutani T, Yoshimatsu K, Arikawa J, Takashima I. 2000. In vitro antiviral activity of lactoferrin and ribavirin upon hantavirus. Arch. Virol. 145:1571-1582.
Norris G E, Gartner A L, Anderson B F, Ward J, Baker E N, Rumball S V, Baker H M. 1986. Preliminary crystallographic studies on bovine lactoferrin. J. Mol. Biol. 191:143-145.
Pietrantoni A, Di Biase A M, Tinari A, Marchetti M, Valenti P, Seganti L, Superti F. 2003. Bovine lactoferrin inhibits adenovirus infection by interacting with viral structural polypeptides. Antimicrob. Agents Chemother. 47:2688-91.
Pietrantoni A, Ammendolia M G, Tinari A, Siciliano R, Valenti P, Superti F. 2006. Bovine lactoferrin peptidic fragments involved in inhibition of Echovirus 6 in vitro infection. Antiviral Res 69:98-106.
Pietrantoni A, Dofrelli E, Tinari A, Ammendolia M G, Puzelli S, Fabiani C, Donatelli I, Superti F. 2010. Bovine lactoferrin inhibits influenza A virus induced programmed cell death in vitro. Biometals 23:465-475.
Ruigrok R W, Wrigley N G, Calder L J, Cusack S, Wharton S A, Brown E B, Skehel J J. 1986. Electron microscopy of the low pH structure of influenza virus haemagglutinin. EMBO J. 5:41-49.
Sano H, Nagai K, Tsutsumi H, Kuroki K. 2003. Lactoferrin and surfactant protein A exhibit distinct binding specificity to F protein and differently modulate respiratory syncytial virus infection. Eur. J. Immunol. 33:2894-2902.
Skehel J J, Wiley D C. 2000. Receptor binding and membrane fusion in virus entry: the influenza hemagglutinin. Annu. Rev. Biochem. 69:531-569.
Smith J R, Rayner C R, Donner B, Wollenhaupt M, Klumpp K, Dutkowski R. 2011 Oseltamivir in seasonal, pandemic, and avian influenza: a comprehensive review of 10-years clinical experience. Adv Ther. 28:927-959.
Steijns J M, van Hooijdonk A C. 2000. Occurrence, structure, biochemical properties and technological characteristics of lactoferrin. Br. J. Nutr. 84 Suppl 1:S11-17.
Sui J, Hwang W C, Perez S, Wei G, Aird D, Chen L M, Santelli E, Stec B, Cadwell G, Ali M, Wan H, Murakami A, Yammanuru A, Han T, Cox N J, Bankston L A, Donis R O, Liddington R C, Marasco W A. 2009. Structural and functional bases for broad-spectrum neutralization of avian and human influenza A viruses. Nat. Struct. Mol. Biol. 16:265-273.
Superti F, Ammendolia M G, Valenti P, Seganti L. 1997. Antirotaviral activity of milk proteins: lactoferrin prevents rotavirus infection in the enterocyte-like cell line HT-29. Med. Microbiol. Immunol. 186:83-91.
Superti F, Siciliano R, Rega B, Giansanti F, Valenti P, Antonini G, 2001. Involvement of bovine lactoferrin metal saturation, sialic acid and protein fragments in the inhibition of rotavirus infection. Biochim. Biophys. Acta 1528:107-115.
Swart P J, Kuipers M E, Smit C, Pauwels R, de B'ethune M P, DeClercq E, Meijer D K F, Huisman J G. 1996. Antiviral effects of milk proteins: acylation results in polyanionic compounds with potent activity against human immunodeficiency virus types 1 and 2 in vitro. AIDS Res. Hum. Retroviruses 12:769-775.
Tinari A, Pietrantoni A, Ammendolia M G, Valenti, P, Superti F. 2005. Inhibitory activity of bovine lactoferrin against echovirus induced programmed cell death in vitro. Int. J. Antimicrob. Agents 25:433-438.
Valenti P, Antonini G. 2005. Lactoferrin: an important host defence against microbial and viral attack. Cell. Mol. Life Sci. 62:2576-2587.
Waarts B L, Aneke O J, Smit J M, Kimata K, Bittman R, Meijer D K, Wilschut J. 2005. Antiviral activity of human lactoferrin: inhibition of alphavirus interaction with heparan sulfate. Virology 333:284-292.
Wiley D C, Skehel J J. 1987 The structure and function of the hemagglutinin membrane glycoprotein of influenza virus. Annu. Rev. Biochem. 56:365-394.
Wilson I A, Skehel J J, Wiley D C. 1981. Structure of the hemagglutinin membrane glycoprotein of influenza virus at 3 Å resolution. Nature 289:366-373

1. A peptide of lactoferrin C-lobe, or a homolog peptide thereof wherein at least one amino acid is substituted with a corresponding homolog amino acid, or a fragment of said peptide or said homolog peptide, or a mixture of at least one of said peptide and/or said homolog peptide and/or said fragment, said peptide being a solvent exposed loop region of lactoferrin C-lobe and having a percentage identity lower than 33% with respect to a corresponding peptide of lactoferrin N-lobe after optimal alignment; wherein said fragment of said peptide or homolog peptide is different from CVLRP (SEQ ID NO: 13), VLRP (SEQ ID NO: 14), CVL, RP, VL, or AKLGGRPTYEE (SEQ ID NO: 15).

2. The peptide of lactoferrin C-lobe, homolog peptide thereof, fragment of said peptide or homolog peptide or mixture of at least one of said peptide and/or said homolog peptide and/or said fragment according to claim 1, wherein said peptide is chosen from the group consisting of SKHSSLDCVLRP (SEQ ID NO: 1), AGDDQGLDKCVPNSKEK (SEQ ID NO: 2), NGESSADWAKN (SEQ ID NO: 4), NGESTADWAKN (SEQ ID No: 3), KANEGLTWNSLKDK (SEQ ID NO: 8), TGSCAFDEFFSQSCAPGADPKSR (SEQ ID NO: 9), GKNGKNCPDKFC (SEQ ID NO: 10), KSETKN (SEQ ID NO: 11), NDNTECLAKLGGRPTYEE (SEQ ID NO: 12).

3. A medicament comprising the peptide of lactoferrin C-lobe, homolog peptide thereof, fragment of said peptide or homolog peptide or mixture of at least one of said peptides and/or said homolog peptide and/or said fragment as defined in claim 1.

4. A pharmaceutical composition comprising the peptide of lactoferrin C-lobe, homolog peptide thereof, fragment of said peptide or homolog peptide or mixture of at least one of said peptide and/or said homolog peptide and/or said fragment, as defined in claim 1, as active principle, together with one or more pharmaceutically acceptable excipients and/or adjuvants.

5. A method to treat influenza virus infection in a individual, the method comprises
administering to the individual a peptide of lactoferrin C-lobe, or of a homolog peptide thereof wherein at least one amino acid is substituted with a corresponding homolog amino acid, or of a fragment of said peptide or homolog peptide, or of a mixture of at least one of said peptide and/or said homolog peptide and/or said fragment, or a lactoferrin C-lobe or of mixtures of said lactoferrin C-lobe with at least one of said peptide and/or said homolog peptide and/or said fragment, or the pharmaceutical composition as defined in claim 4, in an effective amount to treat the Influenza virus infection in the individual.
wherein said peptide is a solvent exposed loop region of lactoferrin C-lobe and has percentage identity lower than 33% with respect to a corresponding peptide of lactoferrin N-lobe after optimal alignment.

6. The method according to claim 5, wherein said peptide comprises or consists of a peptide chosen from the group consisting of SKHSSLDCVLRP (SEQ ID NO: 1), AGDDQGLDKCVPNSKEK (SEQ ID NO: 2), NGESSADWAKN (SEQ ID NO: 4), NGESTADWAKN (SEQ ID No: 3), KANEGLTWNSLKDK (441-454) (SEQ ID NO: 8), TGSCAFDEFFSQSCAPGADPKSR (478-501) (SEQ ID NO: 9), GKNGKNCPDKFC (619-630) (SEQ ID NO: 10), KSETKN (633-637) (SEQ ID NO: 11), NDNTECLAKLGGRPTYEE (642-659) (SEQ ID NO: 12).

7. The method according to claim 5,
wherein said lactoferrin C-lobe is a bovine lactoferrin C-lobe consisting of the SEQ ID No:5:
YTRVVWCAVGPEEQKKCQQWSQQSGQNVTCATASTTDDCIVLVLKGEADA
LNLDGGYIYTAGKCGLVPVLAENRKSSKHSSLDCVLRPTEGYLAVAVVKK
ANEGLTWNSLKDKKSCHTAVDRTAGWNIPMGLIVNQTGSCAFDEFFSQSC
APGADPKSRLCALCAGDDQGLDKCVPNSKEKYYGYTGAFRCLAEDVGDVA
FVKNDTVWENTNGESTADWAKNLNREDFRLLCLDGTRKPVTEAQSCHLAV
APNHAVVSRSDRAAHVKQVLLHQQALFGKNGKNCPDKFCLFKSETKNLLF
NDNTECLAKLGGRPTYEEYLGTEYVTAIANLKKCSTSPLLEACAFLTR

8. The method according to claim 5, wherein Influenza virus infection is type A Influenza virus infection.

9. The method according to claim 8, wherein the type A Influenza virus infection is chosen from the group consisting of H1, H2, H5, H6, H8, H9, H11, H12, H13, H16, H3, H4, H7, H10, H14, and H15 subtypes virus infection.

10. A method to study evolution of influenza virus and receptor-binding interaction, the method comprising
using a peptide of lactoferrin C-lobe, homolog peptide thereof wherein at least one amino acid is substituted with a corresponding homolog amino acid, fragment of said peptide or homolog peptide, wherein said peptide is a solvent exposed loop region of lactoferrin C-lobe and has percentage of identity lower than 33% with respect to a corresponding peptide of lactoferrin N-lobe after optimal alignment.

11. The method according to claim 10, wherein said peptide comprises or consists of a peptide chosen from the group consisting of SKHSSLDCVLRP (SEQ ID NO: 1), AGDDQGLDKCVPNSKEK (SEQ ID NO: 2), NGESSADWAKN (SEQ ID NO: 4), NGESTADWAKN (SEQ ID No: 3), KANEGLTWNSLKDK (441-454) (SEQ ID NO: 8), TGSCAFDEFFSQSCAPGADPKSR (478-501) (SEQ ID NO: 9), GKNGKNCPDKFC (619-630) (SEQ ID NO: 10), KSETKN (633-637) (SEQ ID NO: 11), NDNTECLAKLGGRPTYEE (642-659) (SEQ ID NO: 12).

12. A method to treat an individual, the method comprising
administering to the individual peptide of lactoferrin C-lobe, homolog peptide thereof, fragment of said peptide or homolog peptide or mixture of at least one of said peptides and/or said homolog peptide and/or said fragment as defined in claim 1

13. The pharmaceutical composition according to claim 4, wherein said peptide comprises or consists of a peptide chosen from the group consisting of SKHSSLDCVLRP (SEQ ID NO: 1), AGDDQGLDKCVPNSKEK (SEQ ID NO: 2), NGESSADWAKN (SEQ ID NO: 4), NGESTADWAKN (SEQ ID No: 3), KANEGLTWNSLKDK (441-454) (SEQ ID NO: 8), TGSCAFDEFFSQSCAPGADPKSR (478-501) (SEQ ID NO: 9), GKNGKNCPDKFC (619-630) (SEQ ID NO: 10), KSETKN (633-637) (SEQ ID NO: 11), NDNTECLAKLGGRPTYEE (642-659) (SEQ ID NO: 12).

14. The pharmaceutical composition according to claim 4, wherein said lactoferrin C-lobe is a bovine lactoferrin C-lobe consisting of SEQ ID No:5:
YTRVVWCAVGPEEQKKCQQWSQQSGQNVTCATASTTDDCIVLVLKGEADA
LNLDGGYIYTAGKCGLVPVLAENRKSSKHSSLDCVLRPTEGYLAVAVVKK
ANEGLTWNSLKDKKSCHTAVDRTAGWNIPMGLIVNQTGSCAFDEFFSQSC
APGADPKSRLCALCAGDDQGLDKCVPNSKEKYYGYTGAFRCLAEDVGDVA
FVKNDTVWENTNGESTADWAKNLNREDFRLLCLDGTRKPVTEAQSCHLAV
APNHAVVSRSDRAAHVKQVLLHQQALFGKNGKNCPDKFCLFKSETKNLLF
NDNTECLAKLGGRPTYEEYLGTEYVTAIANLKKCSTSPLLEACAFLTR
